SEQUENCE LISTING (100 sequences)

SEQ ID NO: 1 - protein, 6 aa, Artificial Sequence
  Commercially obtained or synthesized polypeptide obtained by known synthetic methods
AAVIQL

SEQ ID NO: 2 - protein, 11 aa, Artificial Sequence
  Commercially obtained or synthesized polypeptide obtained by known synthetic methods
ADESKHVWSQT

SEQ ID NO: 3 - protein, 7 aa, Artificial Sequence
  Commercially obtained or synthesized polypeptide obtained by known synthetic methods
AFTQGLK

SEQ ID NO: 4 - protein, 13 aa, Artificial Sequence
  Commercially obtained or synthesized polypeptide obtained by known synthetic methods
AGTFSTPRKKFKK

SEQ ID NO: 5 - protein, 14 aa, Artificial Sequence
  Commercially obtained or synthesized polypeptide obtained by known synthetic methods
AGTVLIEDNNFTNE

SEQ ID NO: 6 - protein, 8 aa, Artificial Sequence
  Commercially obtained or synthesized polypeptide obtained by known synthetic methods
ATCESRWT

SEQ ID NO: 7 - protein, 12 aa, Artificial Sequence
  Commercially obtained or synthesized polypeptide obtained by known synthetic methods
ATPSILQTPKTT

SEQ ID NO: 8 - protein, 9 aa, Artificial Sequence
  Commercially obtained or synthesized polypeptide obtained by known synthetic methods
AVLTEEDSD

SEQ ID NO: 9 - protein, 14 aa, Artificial Sequence
  Commercially obtained or synthesized polypeptide obtained by known synthetic methods
DDEENQSLTTKKES

SEQ ID NO: 10 - protein, 13 aa, Artificial Sequence
  Commercially obtained or synthesized polypeptide obtained by known synthetic methods
DDENDSYTDHENI

SEQ ID NO: 11 - protein, 9 aa, Artificial Sequence
  Commercially obtained or synthesized polypeptide obtained by known synthetic methods
DDTDEIEND

SEQ ID NO: 12 - protein, 7 aa, Artificial Sequence
  Commercially obtained or synthesized polypeptide obtained by known synthetic methods
DEENSQT

SEQ ID NO: 13 - protein, 15 aa, Artificial Sequence
  Commercially obtained or synthesized polypeptide obtained by known synthetic methods
DEGESTQSVKTPRKK

SEQ ID NO: 14 - protein, 6 aa, Artificial Sequence
  Commercially obtained or synthesized polypeptide obtained by known synthetic methods
DELHSA

SEQ ID NO: 15 - protein, 10 aa, Artificial Sequence
  Commercially obtained or synthesized polypeptide obtained by known synthetic methods
DENTSENQSE

SEQ ID NO: 16 - protein, 8 aa, Artificial Sequence
  Commercially obtained or synthesized polypeptide obtained by known synthetic methods
DENVEDDE

SEQ ID NO: 17 - protein, 7 aa, Artificial Sequence
  Commercially obtained or synthesized polypeptide obtained by known synthetic methods
DNEVADN

SEQ ID NO: 18 - protein, 12 aa, Artificial Sequence
  Commercially obtained or synthesized polypeptide obtained by known synthetic methods
DYTQMPISWKRK

SEQ ID NO: 19 - protein, 15 aa, Artificial Sequence
  Commercially obtained or synthesized polypeptide obtained by known synthetic methods
EDEETEQSLPKKEED

SEQ ID NO: 20 - protein, 11 aa, Artificial Sequence
  Commercially obtained or synthesized polypeptide obtained by known synthetic methods
EDHWNDPRSAV

SEQ ID NO: 21 - protein, 10 aa, Artificial Sequence
  Commercially obtained or synthesized polypeptide obtained by known synthetic methods
EDNRTPSTAI

SEQ ID NO: 22 - protein, 15 aa, Artificial Sequence
  Commercially obtained or synthesized polypeptide obtained by known synthetic methods
EDNTQVIPRKSLTWS

SEQ ID NO: 23 - protein, 13 aa, Artificial Sequence
  Commercially obtained or synthesized polypeptide obtained by known synthetic methods
EDSYTQSLPKKTS

SEQ ID NO: 24 - protein, 14 aa, Artificial Sequence
  Commercially obtained or synthesized polypeptide obtained by known synthetic methods
EDTSTENKNTNDEE

SEQ ID NO: 25 - protein, 11 aa, Artificial Sequence
  Commercially obtained or synthesized polypeptide obtained by known synthetic methods
EKHSYTNLSPR

SEQ ID NO: 26 - protein, 10 aa, Artificial Sequence
  Commercially obtained or synthesized polypeptide obtained by known synthetic methods
EKSTANPSQD

SEQ ID NO: 27 - protein, 6 aa, Artificial Sequence
  Commercially obtained or synthesized polypeptide obtained by known synthetic methods
ELGQNS

SEQ ID NO: 28 - protein, 9 aa, Artificial Sequence
  Commercially obtained or synthesized polypeptide obtained by known synthetic methods
ENDTHMENS

SEQ ID NO: 29 - protein, 9 aa, Artificial Sequence
  Commercially obtained or synthesized polypeptide obtained by known synthetic methods
ENSADNDEL

SEQ ID NO: 30 - protein, 12 aa, Artificial Sequence
  Commercially obtained or synthesized polypeptide obtained by known synthetic methods
ESEDDMVNTDEE

SEQ ID NO: 31 - protein, 6 aa, Artificial Sequence
  Commercially obtained or synthesized polypeptide obtained by known synthetic methods
GAYNYE

SEQ ID NO: 32 - protein, 9 aa, Artificial Sequence
  Commercially obtained or synthesized polypeptide obtained by known synthetic methods
GNTRKVEVR

SEQ ID NO: 33 - protein, 13 aa, Artificial Sequence
  Commercially obtained or synthesized polypeptide obtained by known synthetic methods
IFTAYQSPRKSTI

SEQ ID NO: 34 - protein, 10 aa, Artificial Sequence
  Commercially obtained or synthesized polypeptide obtained by known synthetic methods
ISLTQPKRFW

SEQ ID NO: 35 - protein, 14 aa, Artificial Sequence
  Commercially obtained or synthesized polypeptide obtained by known synthetic methods
IVRKSATNSLPKKV

SEQ ID NO: 36 - protein, 15 aa, Artificial Sequence
  Commercially obtained or synthesized polypeptide obtained by known synthetic methods
KKETQFKRSTKQSLS

SEQ ID NO: 37 - protein, 8 aa, Artificial Sequence
  Commercially obtained or synthesized polypeptide obtained by known synthetic methods
KKFSQLLK

SEQ ID NO: 38 - protein, 12 aa, Artificial Sequence
  Commercially obtained or synthesized polypeptide obtained by known synthetic methods
KKRKKKTMIKSK

SEQ ID NO: 39 - protein, 13 aa, Artificial Sequence
  Commercially obtained or synthesized polypeptide obtained by known synthetic methods
KKRSLIKKSRPKS

SEQ ID NO: 40 - protein, 14 aa, Artificial Sequence
  Commercially obtained or synthesized polypeptide obtained by known synthetic methods
KKRSTSTQLVKRRT

SEQ ID NO: 41 - protein, 7 aa, Artificial Sequence
  Commercially obtained or synthesized polypeptide obtained by known synthetic methods
KKRTRLK

SEQ ID NO: 42 - protein, 13 aa, Artificial Sequence
  Commercially obtained or synthesized polypeptide obtained by known synthetic methods
KKTRSTLQRKIRK

SEQ ID NO: 43 - protein, 6 aa, Artificial Sequence
  Commercially obtained or synthesized polypeptide obtained by known synthetic methods
KRAKRR

SEQ ID NO: 44 - protein, 8 aa, Artificial Sequence
  Commercially obtained or synthesized polypeptide obtained by known synthetic methods
KRQSIHSA

SEQ ID NO: 45 - protein, 12 aa, Artificial Sequence
  Commercially obtained or synthesized polypeptide obtained by known synthetic methods
KRSKRTKSPKIS

SEQ ID NO: 46 - protein, 11 aa, Artificial Sequence
  Commercially obtained or synthesized polypeptide obtained by known synthetic methods
KRWTGCALRKR

SEQ ID NO: 47 - protein, 6 aa, Artificial Sequence
  Commercially obtained or synthesized polypeptide obtained by known synthetic methods
LENQEI

SEQ ID NO: 48 - protein, 15 aa, Artificial Sequence
  Commercially obtained or synthesized polypeptide obtained by known synthetic methods
LITASFTQSLPRKSG

SEQ ID NO: 49 - protein, 11 aa, Artificial Sequence
  Commercially obtained or synthesized polypeptide obtained by known synthetic methods
MAFMTQSVHVT

SEQ ID NO: 50 - protein, 8 aa, Artificial Sequence
  Commercially obtained or synthesized polypeptide obtained by known synthetic methods
MAVENDES

SEQ ID NO: 51 - protein, 13 aa, Artificial Sequence
  Commercially obtained or synthesized polypeptide obtained by known synthetic methods
MEDMEHSENTEIT

SEQ ID NO: 52 - protein, 9 aa, Artificial Sequence
  Commercially obtained or synthesized polypeptide obtained by known synthetic methods
MFSTQTLKR

SEQ ID NO: 53 - protein, 7 aa, Artificial Sequence
  Commercially obtained or synthesized polypeptide obtained by known synthetic methods
MGHVQSL

SEQ ID NO: 54 - protein, 12 aa, Artificial Sequence
  Commercially obtained or synthesized polypeptide obtained by known synthetic methods
MGTWTQISLPRK

SEQ ID NO: 55 - protein, 10 aa, Artificial Sequence
  Commercially obtained or synthesized polypeptide obtained by known synthetic methods
MITQLIPRMS

SEQ ID NO: 56 - protein, 6 aa, Artificial Sequence
  Commercially obtained or synthesized polypeptide obtained by known synthetic methods
MLSQTI

SEQ ID NO: 57 - protein, 10 aa, Artificial Sequence
  Commercially obtained or synthesized polypeptide obtained by known synthetic methods
MQTISPTARE

SEQ ID NO: 58 - protein, 13 aa, Artificial Sequence
  Commercially obtained or synthesized polypeptide obtained by known synthetic methods
MQTSSYIALTMSM

SEQ ID NO: 59 - protein, 7 aa, Artificial Sequence
  Commercially obtained or synthesized polypeptide obtained by known synthetic methods
MSTAVLA

SEQ ID NO: 60 - protein, 12 aa, Artificial Sequence
  Commercially obtained or synthesized polypeptide obtained by known synthetic methods
NDEHDEHKRVKT

SEQ ID NO: 61 - protein, 8 aa, Artificial Sequence
  Commercially obtained or synthesized polypeptide obtained by known synthetic methods
NDSQLDKT

SEQ ID NO: 62 - protein, 16 aa, Artificial Sequence
  Commercially obtained or synthesized polypeptide obtained by known synthetic methods
NEDDDEFSSSPRKKTS

SEQ ID NO: 63 - protein, 6 aa, Artificial Sequence
  Commercially obtained or synthesized polypeptide obtained by known synthetic methods
NEIDEG

SEQ ID NO: 64 - protein, 13 aa, Artificial Sequence
  Commercially obtained or synthesized polypeptide obtained by known synthetic methods
NEMVLTQSHNEDE

SEQ ID NO: 65 - protein, 11 aa, Artificial Sequence
  Commercially obtained or synthesized polypeptide obtained by known synthetic methods
NEYILDQTLED

SEQ ID NO: 66 - protein, 15 aa, Artificial Sequence
  Commercially obtained or synthesized polypeptide obtained by known synthetic methods
NKASIEEDNDPNIRS

SEQ ID NO: 67 - protein, 14 aa, Artificial Sequence
  Commercially obtained or synthesized polypeptide obtained by known synthetic methods
NMCTQNLLRKTMSE

SEQ ID NO: 68 - protein, 9 aa, Artificial Sequence
  Commercially obtained or synthesized polypeptide obtained by known synthetic methods
NNDECWSAT

SEQ ID NO: 69 - protein, 10 aa, Artificial Sequence
  Commercially obtained or synthesized polypeptide obtained by known synthetic methods
NNSPSEETEA

SEQ ID NO: 70 - protein, 7 aa, Artificial Sequence
  Commercially obtained or synthesized polypeptide obtained by known synthetic methods
NVRKKLK

SEQ ID NO: 71 - protein, 15 aa, Artificial Sequence
  Commercially obtained or synthesized polypeptide obtained by known synthetic methods
RAKRITKFTQSIPKK

SEQ ID NO: 72 - protein, 9 aa, Artificial Sequence
  Commercially obtained or synthesized polypeptide obtained by known synthetic methods
RGKKLHRTV

SEQ ID NO: 73 - protein, 10 aa, Artificial Sequence
  Commercially obtained or synthesized polypeptide obtained by known synthetic methods
RIKRRSYSTS

SEQ ID NO: 74 - protein, 10 aa, Artificial Sequence
  Commercially obtained or synthesized polypeptide obtained by known synthetic methods
RISKKRTYST

SEQ ID NO: 75 - protein, 10 aa, Artificial Sequence
  Commercially obtained or synthesized polypeptide obtained by known synthetic methods
RKKSKAVKKI

SEQ ID NO: 76 - protein, 13 aa, Artificial Sequence
  Commercially obtained or synthesized polypeptide obtained by known synthetic methods
RKSRKLIYHKMKK

SEQ ID NO: 77 - protein, 7 aa, Artificial Sequence
  Commercially obtained or synthesized polypeptide obtained by known synthetic methods
RKVSQLT

SEQ ID NO: 78 - protein, 11 aa, Artificial Sequence
  Commercially obtained or synthesized polypeptide obtained by known synthetic methods
RRQSLLTKKAR

SEQ ID NO: 79 - protein, 12 aa, Artificial Sequence
  Commercially obtained or synthesized polypeptide obtained by known synthetic methods
RSTIRTHQLKKR

SEQ ID NO: 80 - protein, 6 aa, Artificial Sequence
  Commercially obtained or synthesized polypeptide obtained by known synthetic methods
RVHYKK

SEQ ID NO: 81 - protein, 15 aa, Artificial Sequence
  Commercially obtained or synthesized polypeptide obtained by known synthetic methods
SAKISKKRSSKPSAV

SEQ ID NO: 82 - protein, 7 aa, Artificial Sequence
  Commercially obtained or synthesized polypeptide obtained by known synthetic methods
SATLAHI

SEQ ID NO: 83 - protein, 14 aa, Artificial Sequence
  Commercially obtained or synthesized polypeptide obtained by known synthetic methods
SMMSTLYSWSEDMT

SEQ ID NO: 84 - protein, 14 aa, Artificial Sequence
  Commercially obtained or synthesized polypeptide obtained by known synthetic methods
SSVTQSLGVIHFYS

SEQ ID NO: 85 - protein, 8 aa, Artificial Sequence
  Commercially obtained or synthesized polypeptide obtained by known synthetic methods
STASDHSS

SEQ ID NO: 86 - protein, 8 aa, Artificial Sequence
  Commercially obtained or synthesized polypeptide obtained by known synthetic methods
STAVRRSL

SEQ ID NO: 87 - protein, 15 aa, Artificial Sequence
  Commercially obtained or synthesized polypeptide obtained by known synthetic methods
SVGLITQSSLPKKSV

SEQ ID NO: 88 - protein, 12 aa, Artificial Sequence
  Commercially obtained or synthesized polypeptide obtained by known synthetic methods
TGTSLQHYQSSL

SEQ ID NO: 89 - protein, 10 aa, Artificial Sequence
  Commercially obtained or synthesized polypeptide obtained by known synthetic methods
TIAVYTPRKS

SEQ ID NO: 90 - protein, 14 aa, Artificial Sequence
  Commercially obtained or synthesized polypeptide obtained by known synthetic methods
TKKRKITQSPEERK

SEQ ID NO: 91 - protein, 8 aa, Artificial Sequence
  Commercially obtained or synthesized polypeptide obtained by known synthetic methods
TTQSIKTI

SEQ ID NO: 92 - protein, 11 aa, Artificial Sequence
  Commercially obtained or synthesized polypeptide obtained by known synthetic methods
TWSAVHSPQST

SEQ ID NO: 93 - protein, 14 aa, Artificial Sequence
  Commercially obtained or synthesized polypeptide obtained by known synthetic methods
VASTSTQSLPTSWS

SEQ ID NO: 94 - protein, 6 aa, Artificial Sequence
  Commercially obtained or synthesized polypeptide obtained by known synthetic methods
VGTQSI

SEQ ID NO: 95 - protein, 11 aa, Artificial Sequence
  Commercially obtained or synthesized polypeptide obtained by known synthetic methods
VKKRSRSKKKL

SEQ ID NO: 96 - protein, 10 aa, Artificial Sequence
  Commercially obtained or synthesized polypeptide obtained by known synthetic methods
VQSAWCTSAD

SEQ ID NO: 97 - protein, 9 aa, Artificial Sequence
  Commercially obtained or synthesized polypeptide obtained by known synthetic methods
VSIEDNTEA

SEQ ID NO: 98 - protein, 8 aa, Artificial Sequence
  Commercially obtained or synthesized polypeptide obtained by known synthetic methods
VSMENQSA

SEQ ID NO: 99 - protein, 12 aa, Artificial Sequence
  Commercially obtained or synthesized polypeptide obtained by known synthetic methods
VSQLSTSQLLTS

SEQ ID NO: 100 - protein, 7 aa, Artificial Sequence
  Commercially obtained or synthesized polypeptide obtained by known synthetic methods
VTSLRRA

(19) United States
(12) Patent Application Publication
(10) Pub. No.: US 2008/0227716 A1
(43) Pub. Date: Sep. 18, 2008

(54) Use of Peptides for Protecting Skin from Hair-Treatment Agents

(76) Inventors: Helga Rothe, Darmstadt (DE); Veronique Detappe, Fribourg (CH); Friedrich Noser, Bonnefontaine (CH)

Correspondence Address: Michael J Striker;STRIKER STRIKER & STENBY, 103 East Neck Road, Huntington, NY, 11743, US

(21) Appl. No.: 10/580,744

(22) PCT Filed: Nov. 11, 2004
(86) PCT No.: PCT/EP04/12768

§ 371 Date: Feb. 16, 2007

(30) Foreign Application Priority Data
Nov. 28, 2003 (DE) 103 55 743.1

(51) Int. Cl. A61K 38/04; A61Q 17/00

(52) U.S. Cl. 514/14; 514/15; 514/16; 514/17

(57) ABSTRACT

The present invention relates to the use of short-chain peptides for the protection of the skin from hair-treatment agents, and particularly for the protection of the scalp from dyes or colorants.

The present invention concerns the use of short-chain peptides for protecting the skin from hair-treatment agents, particularly for protecting the scalp from dyes or colorants which are known to be used for dyeing hair.

In the treatment of hair with a cosmetic agent, skin contact with the agent cannot always be avoided even when the most careful procedures are used. Such skin contact represents a special drawback in the use of hair colorants. In the main visible regions directly adjacent to the hair roots, such skin staining has a deleterious effect and should be removed or prevented in advance.

It is known to prevent this drawback by use of various emulsions as skin protectants before employing hair-treatment agents. In this case, however, the desired effect is attained only insufficiently, particularly in the use of hair colorants.

Other agents, for example vaseline, must be removed after the hair treatment and, in addition, they have the drawback that because of their insolubility in water they are very difficult to remove from the skin and hair roots or possibly require the use of a skin-irritating solvent for this purpose.

Our goal therefore was to provide preparations intended to prevent skin contact of cosmetic agents, particularly hair colorants, and which would not present the drawbacks of conventional preparations.

Surprisingly, we have now found that the use of a preparation containing a short-chain peptide solves in outstanding manner the problem of protecting skin from undesirable contact with agents for cosmetic treatment of hair, particularly of protecting the skin from undesirable staining during the dyeing or tinting of hair.

Protection of the skin from undesirable contact with agents for cosmetic treatment of hair refers to agents capable of dyeing, tinting, deforming, hardening, conditioning, softening, repairing or styling hair. These effects and the hair-treatment agents causing such effects are known to those skilled in the art.

Hence, the present invention relates to the use of a preparation containing a short-chain peptide for protecting skin from hair-treatment agents.

In particular, the present invention concerns the use of a preparation containing a short-chain peptide for the protection of the skin from hair-treatment agents including hair colorants that can dye, tint, deform, harden, condition, soften, repair or style hair.

More particularly, the present invention concerns the use of a preparation containing a short-chain peptide for protecting skin from hair-treatment agents capable of dyeing or tinting hair and thus possibly staining the skin, particularly the scalp, the said preparation being capable of preventing or reducing such undesirable staining.

By the fact that the use of peptides according to the invention prevents or at least reduces the contact of hair-treatment agents with the skin, undesirable conditions of the skin can be prevented or attenuated. These undesirable conditions comprise, in particular, staining, hypersensitivity reactions, dermatitis, allergic or inflammatory processes and braiding or dandruff formation.

In the dyeing of hair, in particular, the undesirable staining of the part of the head involved presents a special problem for the hairdresser as well as for the private user.

Hence, the use according to the invention for protecting the skin from undesirable skin staining during the dyeing or tinting of hair is particularly preferred.

The short-chain peptides to be used according to the invention have a chain length between 2 and 30 amino acids, preferably between 6 and 15 amino acids and particularly between 6 and 12 amino acids.

In the sense of the present invention, by peptides are meant those the monomers of which consist of amino acids or amino acid derivatives or mixtures thereof that are linked to each other in the usual manner by acid amide bonds. They can be of synthetic (prepared by total industrial synthesis), semisynthetic (obtained by partial synthesis and from natural sources) or natural origin, genetic engineering and microbiological methods of preparation (for example the known methods of DNA recombination) being included.

Also suitable for use are peptides the amino acids of which contain heteroatoms or heteroatom groupings. Suitable amino acid derivatives are, for example, alpha-aminocarboxylic acids with at least three carbon atoms wherein the heteroatoms or heteroatom groups, for example a free amino, hydroxyl, sulfonyl or carboxyl group is preferably linked to the terminal carbon atom.

Hence, the invention also comprises peptides containing amino acid derivatives bearing a free amino, hydroxyl or sulfonyl group.

Particularly preferred are short-chain peptides that are preferably in the form of a dimer or 30-mer, particularly between the trimer and 15-mer and more particularly in the form of a 6-mer to 12-mer. Suitable are, for example, the peptides having the following amino acid sequence (the amino acids are indicated by their known one-letter code):

AAVIQL, ADESKHVWSQT, AFTQGLK, AGTFSTPRKKFKK, AGTVLIEDNNFTNE, ATCESRWT, ATPSILQTPKTT, AVLTEEDSD, DDEENQSLTTKKES, DDENDSYTDHENI, DDTDEIEND, DEENSQT, DEGESTQSVKTPRKK, DELHSA, DENTSENQSE, DENVEDDE, DNEVADN, DYTQMPISWKRK, EDEETEQSLPKKEED, EDHWNDPRSAV, EDNRTPSTAI, EDNTQVIPRKSLTWS, EDSYTQSLPKKTS, EDTSTENKNTNDEE, EKHSYTNLSPR, EKSTANPSQD, ELGQNS, ENDTHMENS, ENSADNDEL, ESEDDMVNTDEE, GAYNYE, GNTRKVEVR, IFTAYQSPRKSTI, ISLTQPKRFW, IVRKSATNSLPKKV, KKETQFKRSTKQSLS, KKFSQLLK, KKRKKKTMIKSK, KKRSLIKKSRPKS, KKRSTSTQLVKRRT, KKRTRLK, KKTRSTLQRKIRK, KRAKRR, KRQSIHSA, KRSKRTKSPKIS, KRWTGCALRKR, LENQEI, LITASFTQSLPRKSG, MAFMTQSVHVT, MAVENDES, MEDMEHSENTEIT, MFSTQTLKR, MGHVQSL, MGTWTQISLPRK, MITQLIPRMS, MLSQTI, MQTISPTARE, MQTSSYIALTMSM, MSTAVLA, NDEHDEHKRVKT, NDSQLDKT, NEDDEFSSSPRKKTS, NEIDEG, NEMVLTQSHNEDE, NEYILDQTLED, NKASIEEDNDPNIRS, NMCTQNLLRKTMSE, NNDECWSAT, NNSPSEETEA, NVRKKLK, RAKRITKFTQSIPKK, RGKKLHRTV, RIKRRSYSTS, RISKKRTYST, RKKSKAVKKI, RKSRKLIYHKMKK, RKVSQLT, RRQSLLTKKAR, RSTIRTHQLKKR, RVHYKK, SAKISKKRSSKPSAV, SATLAHI, SMMSTLYSWSEDMT, SSVTQSLGVIHFYS, STASDHSS, STAVRRSL, SVGLITQSSLPKKSV, TGTSLQHYQSSL, TIAVYTPRKS, TKKRKITQSPEERK, TTQSIKTI, TWSAVHSPQST, VASTSTQSLPTSWS, VGTQSI, VKKRSRSKKKL, VQSAWCTSAD, VSIEDNTEA, VSMENQSA, VSQLSTSQLLTS, VTSLRRA.

The detection and selection of a peptide suitable for the purpose of the present invention can be done by methods that in and of themselves are known and are known to those skilled in the art. Such a method can be carried out in vivo or in vitro and is based on simple comparative testing. Suitable for this purpose are, for example, pigskin preparations the surface of which is treated with a composition containing a peptide of the invention, and using as a control a composition devoid of a peptide. These skin preparations are then brought in contact with a selected cosmetic agent, for example a hair colorant, which after a certain exposure time is again washed out. Thereafter, the amount of the cosmetic agent, for example a dye, left on the skin preparations can be determined quantitatively.

Preferably comprised are peptides which in aqueous solution specifically bind to the skin surface in the pH range between pH 4.0 and pH 8.5 and preferably between pH 5.0 and pH 6.0.

The peptides suitable according to the present invention can be prepared by known methods or obtained commercially, for example from ORPEGEN Pharma, Heidelberg. Thus, a peptide of the desired chain length can by synthesized by a routine method, for example by the generally known Merrifield technique.

Moreover, suitable peptides can be identified by screening of phage peptide libraries (also known as “phage displays”, for example as described by Devlin J J et al., Science 249, 404-406, 1990) or by further optimization via the “Cosmix-Plexing” method according to WO98/33901.

In principle, all known amino acid can be considered for selecting amino acids suitable for the synthesis of the peptide, particularly alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine.

The hair-treatment agents the contact of which is to be prevented or reduced by use of a peptide according to the invention include dyeing or color-imparting, conditioning, deforming, hardening, softening, styling, repairing and/or reconstituting hair-treatment agents.

The said hair-treatment agents include, for example, cationic polymers, cationic surfactants, amidoamines, betaine esters, esterquats, silicone polyols, synthetic polymers, for example acrylic polymers, dyeing or coloring hair-treatment agents including oxidative, nonoxidative, direct, natural, synthetic and semisynthetic dyes, where by direct dyes are meant nitro dyes, azo dyes, quinone dyes, triphenylmethane dyes and acid and basic dyes.

The term hair-treatment agent also comprises the reactive dyes containing a triazinyl, sulfato-ethylsulfonyl or vinylsulfonyl group, for example Reactive Blue 2, Reactive Blue 19, Reactive Red 2, Reactive Orange 16, Reactive Black 5 and Reactive Yellow 2.

According to the invention, the skin is also protected from dye precursors. Dye precursors are, for example, the halogenated nitrobenzene derivatives capable of reacting with compounds having a free amino or hydroxyl group to form nitro dyes. Examples are 4-fluoro-3-nitroaniline, 5-fluoro-2-nitroaniline, 1-chloro-2,4-dinitrobenzene and 1-fluoro-2,4-dinitrobenzene.

Suitable blue nitro dyes are, for example:
1,4-bis[(2′-hydroxyethyl)amino]-2-nitrobenzene,
1-(2′-hydroxyethyl)amino-2-nitro-4-bis-(2″-hydroxyethyl)aminobenzene (HC Blue No. 2),
1-amino-3-methyl-4-(2′-hydroxyethyl)amino-6-nitrobenzene (HC Violet No. 1),
4, N-ethyl, N-(2″-hydroxyethyl)amino-1-(2″-hydroxyethyl)amino-2-nitrobenzene hydrochloride (HC Blue No. 12),
4-bis-(2′-hydroxyethyl)amino-1-(2″-methoxyethyl)amino-2-nitrobenzene (HC Blue No. 11), 1-(2′,3′-dihydroxypropyl)amino-2-nitro-4-[N-methyl-(2″-hydroxyethyl)amino]benzene hydrochloride (HC Blue No. 10),
1-[(2′,3′-dihydroxypropyl)amino]-2-nitro-4-[N-ethyl-(2″-hydroxyethyl)amino]benzene hydrochloride (HC Blue No. 9),
1-(3′-hydroxypropyl)amino-2-nitro-4-bis-(2″-hydroxyethylamino)benzene (HC Violet No. 2), 4, N-methyl, N-(2′,3′-dihydroxypropyl)amino-1-methylamino-2-nitrobenzene hydrochloride (HC Blue No. 6) and
4′-amino-2′-nitro-2″-carboxy-4″-dimethylaminodiphenylamine (HC Blue No. 13).

Suitable red nitro dyes are, for example:
1-amino-4-(2′-hydroxyethyl)amino-2-nitrobenzene (HC Red No. 7),
1-hydroxy-2-amino-4,6-dinitrobenzene,
4-amino-2-nitrodiphenylamine (HC Red No. 1),
1-amino-2-nitro-4-bis-(2′-hydroxyethyl)aminobenzene hydrochloride (HC red No. 13),
1-amino-2-nitro-4-(2′-hydroxyethyl)amino-5-chlorobenzene,
1-(2′-hydroxyethyl)amino-2-nitro-4-aminobenzene (HC Red No. 3),
1-hydroxy-3-nitro-4-aminobenzene,
1-hydroxy-3-nitro-4-(2′-hydroxyethylamino)benzene,
1-(2′-aminoethyl)amino-2-nitro-4-(2′-hydroxethoxy)benzene (HC Orange No. 2),
3-nitro-4-(2′-hydroxyethyl)aminophenylglycerol ether (HC Orange No. 3),
1-amino-5-chloro-4-(2′,3′-dihydroxypropyl)amino-2-nitrobenzene (HC Red No. 10),
1,4-bis-[(2′,3′-dihydroxypropyl)amino]-5-chloro-2-nitrobenzene (HC red. No. 11),
1-hydroxy-2-(2′-hydroxyethyl)amino-4,6-dinitrobenzene,
3-nitro-4-ethylaminobenzoic acid,
4-amino-2-nitrodiphenylamino-2-carboxylic acid,
2-chloro-6-ethylamino-4-nitrophenol,
2-amino-6-chloro-4-nitrophenol,
1-hydroxy-3-nitro-4-(3′-hydroxypropylamino)benzene,
2,5-diamino-6-nitropyridine,
1,2,3,4-tetrahydro-6-nitroquinoxaline and
7-amino-3,4-dihydro-6-nitro-2H-1,4-benzoxazine (HC Red 14).

Suitable yellow nitro dyes are, for example:
1-amino-2-(2′-hydroxyethyl)amino-5-nitrobenzene (HC Yellow No. 5),
1-(2′-hydroxyethoxy)-2-(2″-hydroxyethyl)amino-5-nitrobenzene (HC Yellow No. 4),
1-(2′-hydroxyethyl)amino-2-nitrobenzene (HC Yellow No. 2)
1-methoxy-2-(2′-hydroxyethyl)amino-5-nitrobenzene,
1-hydroxy-2-amino-3-nitrobenzene,
1-amino-2-methyl-6-nitrobenzene,
1-(2′-hydroxyethyl)oxy-3-methylamino-4-nitrobenzene,
1-methylamino-2-nitro-5-(2′,3′-dihydroxypropyl)oxybenzene,
1-(2′-hydroxyethyl)amino-2-hydroxy-4-nitrobenzene (HC Yellow No. 11),
1-methoxy-3-(2′-aminoethyl)amino-4-nitrobenzene hydrochloride (HC Yellow No. 9),
1-(2′-ureidoethyl)amino-4-nitrobenzene,
4-(2′,3′-dihydroxypropyl)amino-3-nitrotrifluoromethylbenzene (HC Yellow No. 6),
2,4-bis-[N-(2′-hydroxyethyl)amino]-5-chloronitrobenzene (HC Yellow No. 10),
4-(2′-hydroxyethyl)amino-3-nitromethylbenzene,
4(2′-hydroxyethyl)amino-3-nitrochlorobenzene (HC Yellow No. 12),
4-(2′-hydroxyethyl)amino-3-nitrotrifluoromethylbenzene (HC Yellow No. 13),
4-(2′-hydroxyethyl)amino-3-nitrobenzonitrile (HC Yellow No. 14),
4-(2′-hydroxyethyl)amino-3-nitrobenzamide (HC Yellow No. 15).

Suitable azo dyes are, for example:
1-(4′-nitrophenylazo)-2-methyl-4-bis-(2′-hydroxyethyl)aminobenzene,
1-(3′-nitro-4-amino)phenylazo-2-hydroxy-7-trimethylammonium naphthalene chloride,
1-(2′-hydroxy-4′-sulfo-6′-nitro)naphthylazo-2-hydroxynaphthalene, CI 15700,
1-(4′-aminophenylazo)-2-methyl-4-bis-[(2′-hydroxyethyl)amino]benzene,
5-(4′-dimethylaminophenylazo)-1,4-dimethyltriazonium chloride,
1-(2′-methoxyphenylazo)-2-hydroxy-7-trimethylammonium naphthalene chloride,
1-(4′-aminophenylazo)-2-hydroxy-7-trimethylammonium naphthalene,
4-(3′-trimethylammoniumphenylazo)-N-phenyl-3-methylpyrazolone (5),
4-hydroxy-3-[(4′-sulfo-1′-naphthyl)azo]-1-naphthalenesulfonic acid,
1-(4′-sulfophenylazo)-2-hydroxynaphthalene,
1-(4′-sulfophenylazo)-2-hydroxy-6-sulfonaphthalene, CI 15985,
4-amino-[4′-bis-(2″-hydroxyethyl)amino]azobenzene,
4-amino-[4′-bis-(2″-hydroxyethyl)amino]-2′-methylazobenzene,
3-(2′,6′-diaminopyridyl-3′-azo)pyridine,
7-phenylazo-1-amino-3,6-disulfo-8-hydroxynaphthalene,
5-acetylamino-4-hydroxy-3-[(2′-methylphenyl)azo]-2,7-naphthalenedisulfonic acid and
2-(2′,4′-dimethylphenylazo)-6-(4″-sulfophenylazo)-1,3-dihydroxybenzene.

Suitable quinone dyes are, for example:
1,4-bis-(2′,3′-dihydroxypropyl)aminoanthraquinone,
1-methylamino-4-(2′-hydroxyethyl)aminoanthraquinone,
2-(2′-aminoethyl)aminoanthraquinone,
2-bromo-4,8-diamino-6-(3′-trimethylammonium)phenylamino-1,5-naphthoquinone,
1-(2′-sulfo-4′-methylphenyl)amino-4-hydroxyanthraquinone,
1,4-diaminoanthraquinone,
1-amino-2-sulfo-4-cyclohexylaminoanthraquinone,
1-methylamino-4-aminopropylaminoanthraquinone,
1-aminopropylaminoanthraquinone,
1,4-diamino-2-methoxyanthraquinone and
1,4-bis-(2-hydroxyethyl)amino-5,8-dihydroxyanthraquinone.

Suitable triphenylmethane dyes are, for example:
4′,4″,4′″-triamino-3-methyltriphenylcarbonium chloride,
40 bis-(4,4-diethylaminophenyl)-4′-ethylaminonaphthylcarbonium chloride,
bis-(4,4-dimethylaminophenyl-4′-phenylaminonaphthylcarbonium chloride, Basic Blue 26, CI 44045, and
4,4-bis-(N-ethyl-3-sulfobenzyl)amino-2″-sulfofuchsonium.

Suitable acid dyes are, for example:
1-(4′-sulfophenylazo)-2-hydroxy-6-sulfonaphthalene, CI 15 985,
1-(2′-hydroxy-4′-sulfo-6′-nitro)naphthylazo-2-hydroxynaphthalene, CI 15 700,
2,4-dinitro-1-naphthol-7-sulfonic acid disodium salt (Acid Yellow 1; CI 10 316),
2-(2′-quinolyl)-1H-indene-1,3(2H)-dione monosulfonic acid disodium salt (Acid Yellow 3; CI 47 005),
4,5-dihydro-5-keto-1-(4′-sulfophenyl)-4-[(4″-sulfophenyl)azo]-1H-pyrazole-3-carboxylic acid trisodium salt (Acid Yellow 23; C119 140),
3′,6′-dihydroxyspiro[isobenzofuran-1(3H), 9′(9H)-xanthen]-3-one (Acid Yellow 73; CI 45 350:1),
5-[2′,4′-dinitrophenyl)amino]-2-(phenylamino)benzenesulfonic acid sodium salt (Acid Orange 3; CI 10 385),
4-[(2′,4′-dihydroxyphenyl)azo]benzenesulfonic acid sodium salt (Acid Orange 6; CI 14 270),
4-[2′-hydroxy-1′-naphthyl)azo]benzenesulfonic acid sodium salt (Acid Orange 7; CI 15 510),
4-[(3′-[(2″,4″-dimethylphenyl)azo]-2′,4′-dihydroxyphenyl)azo]benzenesulfonic acid sodium salt (Acid Orange 24; CI 20 170),
4-hydroxy-3-[(4′-sulfo-1′-naphthyl)azo]-1-naphthalenesulfonic acid disodium salt (Acid Red 14; CI 14 720),
7-hydroxy-8-[(4′-sulfo-1′-naphthyl)azo]-1,3-naphthalenedisulfonic acid trisodium salt (Acid Red 18; CI 16 255),
3-hydroxy-4-[(4′-sulfo-1′-naphthyl)azo]-2,7-naphthalenedisulfonic acid trisodium salt (Acid Red 27; CI 16 185),
5-amino-4-hydroxy-3-phenylazo-2,7-napthalenedisulfonic acid disodium salt (Acid Red 33; CI 17 200),
5-(acetylamino)-4-hydroxy-3-[(2′-methylphenyl)azo]-2,7-naphthalenedisulfonic acid disodium salt (Acid Red 35; CI 18 065),
3′,6′-dihydroxy-2′,4′,5′,7′-tetraiodospiro[isobenzofuran-1(3H), 9′(9H)-xanthen]-3-one disodium salt (Acid Red 51; CI 45 430),
3,6-bis-(diethylamino)-9-(2′,4′-disulfophenyl)xanthylium hydroxide sodium salt (Acid Red 52; CI 45 100),
7-hydroxy-8-{[4′-(phenylazo)phenyl]azo}-1,3-naphthalenedisulfonic acid disodium salt (Acid Red 73; CI 27 290),
2′,4′,5′,7′-tetrabromo-3′,6′-dihydroxyspiro[isobenzofuran-1(3H), 9′(9H)-xanthen]-3-one disodium salt (Acid Red 87; CI 45 380),
2′,4′,5′,7′-tetrabromo-4,5,6,7-tetrachloro-3′,6′-dihydroxyspiro[isobenzofuran-1(3H), 9′(9H)-xanthen]-3-one disodium salt (Acid Red 92; CI 45 410),
3′,6′-dihydroxy-4′,5′-diiodospiro[isobenzofuran-1(3H), 9′(9H)-xanthen]-3-one disodium salt (Acid Red 95; CI 45 425),
Acid Red 195; Acid Blue 9 (CI 42 090),
2,2′-[(9,10-dihydro-9,10-diketo-1,4-anthracenediyl)diimino]-bis-(5-methylbenzenesulfonic acid) disodium salt (Acid Green 25; CI 61 570),
N-[4-[[4′-(dimethylamino)phenyl]-(2″-hydroxy-3″,6″-disulfo-1″-naphthyl)methylene}-2,5-cyclohexadien-1-ylidene]-N-methylmethanaminium hydroxide (Acid Green 50; CI 44 090),
N-[4-[(4′-(diethylamino)phenyl]-(2″,4″-disulfophenyl)methylene]-2,5-cyclohexadien-1-ylidene]-N-ethylethanaminium hydroxide sodium salt (Acid Blue 1; CI 42 045),
N-{4-[(4′-(diethylamino)phenyl]-(5″-hydroxy-2″,4″-disulfophenyl)methylene}-2,5-cyclohexadien-1-ylidene]-N-ethylethanaminium hydroxide calcium salt (Acid Blue 3; CI 42 051),
1-amino-4-(cyclohexylamino)-9,10-dihydro-9,10-diketo-2-anthracenesulfonic acid sodium salt (Acid Blue 62; CI 62 045),
2-(1′,3′-dihydro-3′-keto-5′-sulfo-2′H-indol-2′-ylidene)-2,3-dihydro-3-keto-1H-indole-5-sulfonic acid disodium salt (Acid Blue 74; CI 73 015),
9-(2′-carboxyphenyl)-3-[(2″-methylphenyl)amino]-6-[(2′″-methyl-4′″-sulfophenyl)amino]xanthylium hydroxide sodium salt (Acid Violet 9; CI 45 190),
2-[(9′,10′-dihydro-4′-hydroxy-9′,10′diketo-1′-anthracenyl)amino]-5-methylbenzenesulfonic acid sodium salt (Acid Violet 43; CI 60 730),
3,3′-[sulfonyl-bis-(2-nitro-4,1-phenylene)imino]-bis-[6-(phenylamino)benzene disodium sulfonate (Acid Brown 13; CI 10 410),
4-amino-5-hydroxy-3-[(4′-nitrophenyl)azo]-6-(phenylazo)-2,7-naphthalenedisulfonic acid disodium salt (Acid Black 1; CI 20 470),
3-hydroxy-4-[(2′-hydroxy-1′-naphthyl)azo]-7-nitro-1-naphthalenesulfonic acid sodium salt (Acid Black 52; CI 15 711) and
3-[(2,4-dimethyl-5-sulfophenyl)azo]-4-hydroxy-1-naphthalenesulfonic acid (Ponceau SX, CI 14 700),

Suitable basic dyes are, for example:
bis-(4,4-dimethylaminophenyl)-4′-phenylaminonaphthylcarbonium chloride (Basic Blue 26; CI 44 045),
N-{4-[(4′-diethylamino)phenyl]-[4″-(ethylamino)-1″-naphthyl]methylene}-2,5-cyclohexadien-1-ylidene-N-ethylethanammonium chloride (Basic Blue 7; CI 42 595),
4-[(4′-aminophenyl)-(4′-imino-2′,5′-cyclohexadien-1′-ylidene)methyl]-2-methylaminobenzene hydrochloride (Basic Violet 14; CI 42 510),
4-(acetylamino)-5-hydroxy-6-[[7′-sulfo-4′-[(4″-sulfophenyl)azo]-1′-naphthyl]azo]-1,7-naphthalenedisulfonic acid tetrasodium salt (Brilliant Black 1; CI 28 440),
[8-(p-aminophenyl)azo]-7-hydroxy-2-naphthyl]trimethylammonium chloride (Basic Brown 16; CI 2 250),
[8-[4′-amino-2′-nitrophenyl)azo]-7-hydroxy-2-naphthyl]trimethylammonium chloride (Basic Brown 17; CI 12 251),
7-hydroxy-8-[(2′-methoxyphenyl)azo]-N,N,N-trimethyl-2-naphthylammonium chloride (Basic Red 76; CI 12 245),
3-[(4′-amino-6′-bromo-5′,8′-dihydro-1′-hydroxy-8′-imino-5′-keto-2′-naphthyl)amino]-N,N,N-trimethylammonium chloride (Basic Blue 99; CI 56 059) and
4-(3′-trimethylammoniumphenylazo)-N-phenyl-3-methylpyrazol-5-one (Basic Yellow 57; CI 12 719).

The amount of peptide to be applied to the skin depends on the amount and kind of the hairtreatment agent used. The short-chain peptide is used in the form of an aqueous solution. A ready-to-use aqueous solution can contain from 0.01 to 100 mg of peptide/mL, particularly from 0.1 to 50 mg/mL, more particularly from 0.25 to 40 mg/mL and preferably from 1.0 to 25 mg of peptide/mL.

In addition, the aqueous solution can contain auxiliary agents, particularly antioxidants (for example tocopherol derivatives), complexing agents [for example ethylenediaminetetraacetic acid (EDTA) or diethylenetriaminepentaacetic acid (DTPA), nitriloacetic acid (NTA) or hydroxyethylenediaminetriacetic acid (HEDTA), a buffer (for example a citrate buffer, citrate-phosphate buffer or phosphate buffer), preservatives or antimicrobial agents (for example, a parahydroxybenzoate ester, benzyl alcohol, and butyl, propyl and methylparabens, sodium hydroxymethylaminoacetate, methylisothiazolinone, phenoxyethanol or Quaternium-15), perfumes, moisturizers, (for example dimethicone silicone, lanolin and lanolin alcohols, amino acids, panthenol, sorbitol, glycerol or propylene glycol), viscosity modifiers (for example, methylcellulose, xanthan gum, hydroxyethylcellulose, methylhydroxyethylcellulose, polyvinylpyrrolidone, an acrylic copolymer or carbomers).

The application or formulation of the short-chain peptides can be done in several ways using fluid, viscous, creamy to pasty formulations. Thus, a composition containing a short-chain peptide can be aqueous or in the form of a lotion, gel, paste or cream.

In the event that the skin must not only be protected from hair-treatment agents but in addition is to receive care, the short-chain peptide can be combined with a care agent. Hence, the present invention includes the use of a preparation containing a short-chain peptide for the protection of the skin from hair-treatment agents and simultaneously for skin care.

This double action can be attained by coupling at least one care agent with a short-chain peptide molecule by a known chemical method.

Suitable for this purpose are, for example: protein hydrolyzates (for example from wheat), amidoamines, moisturizers such as lactates (for example cetyl lactate), vitamins or provitamins or vitamin precursors, for example panthenol and derivatives thereof, biotin, tocopherols, sugars for example polysaccharides, oligosaccharides, glucose, fructose or inulin, organic-chemical UV filters, all known UVA, UVB and UVA/UVB filter substances being suitable either alone or in combination with one another, for example the derivatives of dibenzoylmethane (for example Parsol 1789 supplied by Givaudan/Roure, INCI designation butyl methoxydibenzoylmethane), benzylidenecamphor or derivatives thereof, particularly methylbenzylidenecamphor [for example (3-benzylidenecamphor or 3-(4-methylenebenzylidene)-d,l-camphor], derivatives and esters of cin-namic acid, particularly the derivatives and esters of methoxycinnamic acid (for example octyl 4-methoxycinnamate or isopentyl 4-methoxycinnamate), derivatives and esters of benzoic acid particularly of 4-aminobenzoic acids, polyhydroxybenzoic acid (for example methyl polyhydroxybenzoate or propyl polyhydroxybenzoate), esters of salicylic acid [for example (2-ethylhexyl) salicylate or (4-isopropylbenzyl) salicylate], sulfonic acids, benzophenones and derivatives thereof, for example the sulfonic acid derivatives of benzophenones (for example 2-hydroxy-4-methoxyben-zophenone-5-sulfonic acid) as UVB/UVA filters or of the benzimidazoles (for example 2-phenyl-benzimidazole-5-sulfonic acid) and the salts thereof, dibenzoylmethanes or suitable polypeptides, particularly oxygen radical scavengers, for example the known Mn, Fe or Zn peroxide dismutases, as well as tocopherols and vitamins (for example ascorbic acid).

The said care agents can be present in a total amount between 0.001 and 30.0 wt. %, particularly between 0.01 and 25.0 wt. %, more particularly between 0.1 and 15 wt. % and preferably between 0.5 and 10.0 wt. %, based on the amount of the cosmetic agent to be used.

Legend for the figures:

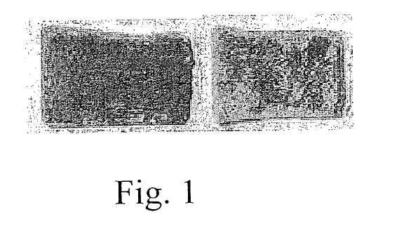

FIG. 1

Pigskin preparations after treatment with a dye solution according to Example 2. The dyeing was done as in Example 4. Before the dyeing, the preparation on the left was pretreated with a peptide solution according to Example 3, while the one on the right was not pretreated in this manner.

The following examples will explain the present invention in greater detail.

### EXAMPLE 1

### Making the Pigskin Preparations

Fresh parts of pigskin (Central-European domestic pig) were thoroughly cleaned with a conventional mild shampoo to remove contamination left from the removal procedure or by hand contact. From these skin specimens, approximately 5.0×5.0 cm parts were cut out and each part was placed in a separate Petri dish.

### EXAMPLE 2

### Preparation of a Dye Solution

A solution having the following composition was prepared

| Raw Material | Amount, wt. % |
|---|---|
| Natrosol 250 HHR¹ | 1.0 |
| Nip Nip² | 0.2 |
| Ethanol (96%) | 5.0 |
| Plantaren 2000 UP³ | 5.0 |
| Demineralized water | 88.8 |
| | 100 |

¹Hydroxyethylcellulose
²Methyl paraben 0.14% + propyl paraben 0.06%
³Decylglucoside (Cognis)

Natrosol 250 HHR and about one third of the water was heated with stirring (magnetic stirrer) to approximately 55° C. until the solution became viscous. Ethanol, Plantaren 2000 UP, Nip Nip and 0.4 wt. % of dye (Basic Violet 14) were mixed with the second third of the water and then brought to a boil. The hot dye solution was added to the Natrosol solution with stirring (magnetic stirrer). The remainder of the water was used to rinse the dye container and was then added to the pre-pared mixture. In a cold water bath, the mixture was then cooled to room temperature with slow stirring (magnetic stirrer).

The pH can be adjusted to 6.0 at about 30° C. with sodium hydroxide solution or citric acid, fluctuations of about pH 0.9 being tolerable.

### EXAMPLE 3

### Treatment of Skin Specimens with a Short-Chain Peptide

We prepared 10-mL portions of an aqueous 5.0 mg/mL solution of a peptide having the sequence LITASFTQSLPRKSG.

Ten mL of this peptide solution was applied with a Pasteur pipette to a skin specimen obtained as in Example 1. The solution was then left on the skin specimen until it dried. Complete drying was achieved within about 10 minutes with the aid of a hair drier.

After 30 minutes, the skin specimens were rinsed with tap water for 2 minutes, placed in a new Petri dish and again dried with a hair drier.

### EXAMPLE 4

### Dyeing the Skin Specimens

The entire surface of the skin specimens treated according to Example 3 as well as of those that were not treated with the short-chain peptide (controls) was covered with the dye solution pre-pared as in Example 2 by use of a brush. After an exposure time of 30 minutes, the skin specimens were rinsed under tap water for 2 minutes while being rubbed with the fingers. The specimens were then transferred to a Petri dish and dried with a hair drier.

The result we found was that the skin specimens treated with the two peptides showed only very minor staining compared to the untreated skin specimens.

1. Use of a preparation containing a short-chain peptide for protecting skin from hair-treatment agents.

2. Use according to claim 1, characterized in that the protection of the skin from hair-treatment agents refers to agents capable of dyeing, tinting, deforming, hardening, conditioning, softening, repairing or styling hair.

3. Use according to claim 1, characterized in that the protection of the skin from hair-treatment agents refers to agents capable of dyeing or tinting hair.

4. Use according to claim 1, characterized in that the short-chain peptide has a chain-length between 2 and 30 amino acids, preferably between 6 and 15 amino acids and particularly between 6 and 12 amino acids.

5. Use according to claim 1 for simultaneously protecting the skin from hair-treatment agents and providing skin care.
